(19) United States
(12) Patent Application Publication
(10) Pub. No.: US 2010/0057116 A1
(43) Pub. Date: Mar. 4, 2010

(54) POROUS MATERIAL CONDUIT AND METHOD FOR PREPARING THE SAME

(75) Inventors: De-Yao Wang, Taipei (TW); Meng-Tze Chen, Taipei (TW); Yi-You Huang, Taipei (TW); Wei-Zen Sun, Taipei (TW)

Correspondence Address: EDWARDS ANGELL PALMER & DODGE LLP, P.O. BOX 55874, BOSTON, MA, 02205, US

(73) Assignee: NATIONAL TAIWAN UNIVERSITY, Taipei (TW)

(21) Appl. No.: 12/351,982

(22) Filed: Jan. 12, 2009

(30) Foreign Application Priority Data
Sep. 2, 2008 (TW) 97133576

(51) Int. Cl. A61B 17/08; B23K 26/00

(52) U.S. Cl. 606/152; 219/121.72

(57) ABSTRACT

The present invention provides a porous material conduit and a method for preparing the same, wherein the porous material conduit includes a porous material basal body and one or more conduit-shaped structures formed in the porous material basal body by laser, and method includes the steps of providing a porous material basal body and injecting a laser beam there into, so as to form a conduit-shaped structure in the porous material basal body to obtain a desired porous material conduit, thereby overcoming the drawback of damages to the porous material basal body caused by an uneven force applied when employing a conventional hole drilling technique. Moreover, a plurality of conduit-shaped structures in a multidirectional intersecting alignment may be easily obtained by adjusting relative displacements and varying the injection angle, of the laser beam with respect to the basal body and of the laser beam.

### BACKGROUND OF THE INVENTION

1. Field of the Invention

The present invention relates generally to porous material conduits and methods for preparing the same, and more particularly to a porous material nerve conduit and a method for preparing the same.

2. Description of Related Art

Nervous regeneration is a very complicated physiological phenomenon. Since mature neurons cannot self-replicate or undergo cell division, it is difficult for the injured neurons to recover to their original states if a nervous system is injured and is not provided with efficient and timely medical care and recovery acts.

Accordingly, various methods are developed for medical treatment and recovery of injured neurons in many of the past clinical and basic researches, wherein in view of the good supportability provided by nerve conduits and their effect of facilitating nerve regeneration, nerve conduits have gradually become a major focus in the fields of biomedical materials and medical engineering.

Nerve conduits suitable for nerve growth are synthesized using a degradable biomedical material, and then they are disposed at injured positions of nerves, such that the injured nerves can be supported by the nerve conduits and cling to insides of the nerve conduits via the pores throughout the nerve conduits, thereby facilitating regeneration and recovery of the injured nerves within a short period of time.

Taiwanese Patent No. I273913 discloses a bionic nerve conduit and a method for preparing the same, wherein a template array is formed by semiconductor lithography, so as to form sub-conduit structures having precise sizes and evenly distributed hollow cavities. Further, through microstructures of the hollow cavities and special designs, injured nerves are guided to cling and grow in specific directions, thereby facilitating rehabilitation of the injured nerves.

Referring to FIG. 1a, FIG. 1a is a schematic diagram showing a hole drilling technique for forming a hollow conduit-shaped structure in a porous nerve conduit that has pores throughout.

However, common nerve conduit basal bodies are quite vulnerable due to the pores found throughout the bodies. Since the hole drilling technique usually requires a porous nerve conduit basal body 10 to be clipped by a clipping element 11 such that a drilling element 12 can drill in a hole in the nerve conduit basal body 10, the porous nerve conduit basal body 10 may be easily damaged when the clipping force applied to clipping positions 131 is uneven or the drilling force applied to a drilling position 132 is excessively large.

To avoid using the hole drilling technique for forming hollow conduit-shaped structures in a vulnerable porous nerve conduit, another conventional approach involves using an injection molding technique to form a desired corresponding structure, performing mold filling to form a porous nerve conduit, and then separating the desired nerve conduit from the mold.

Referring to FIG. 1b, FIG. 1b is a schematic diagram illustrating a conventional mold filling technique on a porous nerve conduit. As shown in FIG. 1b, during the injection molding process, the porous nerve conduit basal body 10 and a corresponding mold 14 are separated in a specific direction, a plurality of hollow conduit-shaped structure arrays formed in the porous nerve conduit by the technique is limited to be a parallel array, instead of having an intersecting alignment.

Therefore, a drawback of the conventional hole drilling technique is that an uneven clipping force or an excessively large drilling force can cause damage to a porous nerve conduit basal body, while a drawback of the conventional injection molding technique is the difficulty in forming a plurality of hollow, intersecting conduit-shaped structures.

Accordingly, it is an urgent issue in the design of nerve conduits and preparations thereof to provide a porous material conduit and a method for preparing the same to overcome the foregoing drawbacks.

### SUMMARY OF THE INVENTION

In order to attain the above ant other objects, it is therefore an object of the present invention to provide a porous material conduit and a method for preparing the same, whereby the drawbacks of the difficulty in forming a plurality of intersecting, hollow conduit-shaped structures by employing the conventional injection molding technique and the damage of a porous nerve conduit basal body caused by an uneven clipping force or an excessively large drilling force when employing the conventional hole drilling technique are overcome.

Therefore, the present invention provides a method for preparing a porous material conduit, which comprises the steps of: providing a porous material basal body; and injecting a laser beam into the porous material basal body, so as to form a conduit-shaped structure in the porous material basal body.

Preferably, the porous material basal body is a chitosan sponge basal body, which is formed by dissolving 2% v/v chitosan in 2% v/v acetic acid solution in a freeze-drying system.

More preferably, during the preparation of the chitosan sponge basal body, the desired pH of the chitosan sponge basal body is obtained by neutralizing with a methanol-sodium hydroxide solution formed by dissolving O.1M sodium hydroxide in 99.9% methanol solution. Then, sodium hydroxide and/or sodium acetate remained on the chitosan sponge basal body is removed by a methanol solution. Finally, residual moisture on the chitosan sponge basal body is removed by a drying method to obtain a desired chitosan sponge basal body.

The laser beam used is preferably a carbon dioxide laser, along with a laser spot adjusted through a lens module, to inject the laser beam into the porous material basal body. In other words, the preferred scenario is to adjust the depth of focus of the laser beam into the porous material basal body by the lens module to control the cross-sectional shape and pattern of the conduit-shaped structure.

The method for preparing a porous material basal body further comprises making relative movement between the laser beam and the porous material basal body in a two-dimensional plane or a three-dimensional space, and then injecting the laser beam into the porous material basal body to form another conduit-shaped structure in the porous material basal body.

Preferably, a plurality of relative movements are generated between the laser beam and the porous material basal body, and the laser beam is injected into the porous material basal body during the intervals between the relative movements, thereby forming a plurality of conduit-shaped structures in the porous material basal body. More preferably, a plurality of relative movements having the same intervals are generated between the laser beam and the porous material basal body to form a plurality of conduit-shaped structures that are parallel arranged into a two-dimensional array.

Moreover, the method further comprises changing the injection angle of the laser beam with respect to the porous material basal body, so that the laser beam into the porous material basal body, thereby forming another conduit-shaped structure in the porous material basal body, wherein original conduit-shaped structure intersect with the another conduit-shaped structures.

More preferably, the injection angle of the leaser beam with respect to the porous material basal body is changed a plurality of times, and the laser beam is injected into the porous material basal body a plurality of times during the changing of each of the injection angles, so as to form a plurality of intersecting, conduit-shaped structures in the porous material basal body. Even more preferably, a plurality of relative movements can be generated between the laser beam and the porous material basal body, and the injection angle of the laser beam with respect to the porous material basal body is changed during the relative movements, so as to form a plurality of conduit-shaped structures in the porous material basal body.

The present invention further provides a porous material conduit comprising a porous material basal body and a conduit-shaped structure formed therein by laser, wherein the porous material basal body is a chitosan sponge basal body, and the laser beam is a carbon dioxide laser.

The present invention further provides a porous material conduit comprising a porous material basal body and a plurality of conduit-shaped structures formed therein, wherein at least a pair of the conduit-shaped structures intersects. Further, the porous material basal body is made of one of a chitosan sponge basal body, hyaluronic acid, collagen, gelatin, algin, polyanhydride, polycaprolacton, polylactic acid and a copolymer thereof, but is not limited thereto (i.e., other porous materials having the same functions can be used as alternatives). The laser beam is a carbon dioxide laser.

In light of the above, the porous material conduit and a method for preparing the same, as disclosed herein, can form a plurality of intersecting, hollow conduit-shaped structures simply by applying laser to a porous material basal body, while overcoming the drawback of the damage to a porous nerve conduit basal body caused by an uneven clipping force or an excessively large drilling force when employing the conventional hole drilling technique.

### BRIEF DESCRIPTION OF THE DRAWINGS

FIG. 1a is a schematic diagram showing a conventional hole drilling technique for forming a porous nerve conduit;

FIG. 1b is a schematic diagram illustrating a conventional mold filling technique on a porous nerve conduit;

FIG. 2a is a first flowchart showing a method of preparing a porous material conduit of the present invention;

FIG. 2b is a second flowchart showing a method of preparing a porous material conduit of the present invention;

FIG. 2c is a third flowchart showing a method of preparing a porous material conduit of the present invention;

FIG. 3a is a first schematic diagram showing a device for preparing a porous material conduit of the present invention;

FIG. 3b is a second schematic diagram showing a device for preparing a porous material conduit of the present invention;

FIG. 3c is a third schematic diagram showing a device for preparing a porous material conduit of the present invention;

FIG. 4a is a first schematic diagram showing a porous material conduit of the present invention;

FIG. 4b is a second schematic diagram showing a porous material conduit of the present invention;

FIG. 4c is a third schematic diagram showing a porous material conduit of the present invention; and

FIG. 5 is a schematic diagram showing the structure of a porous material conduit of the present invention.

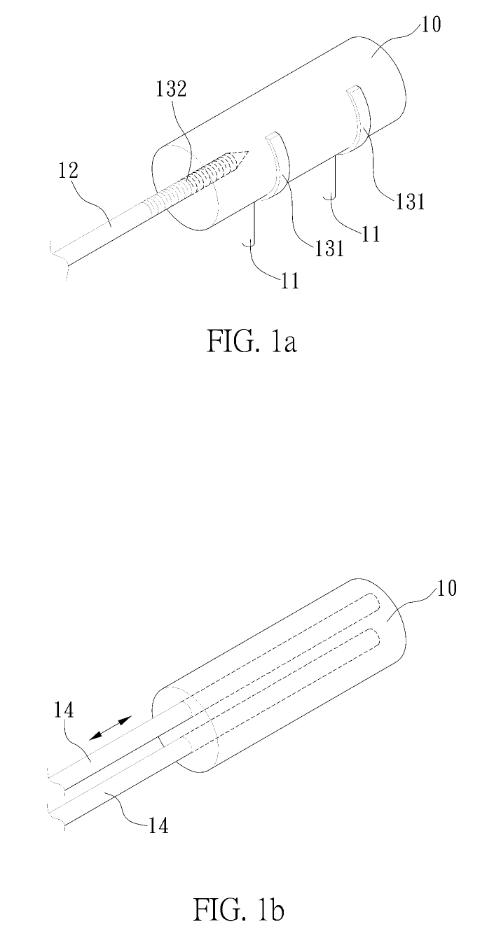

### DETAILED DESCRIPTION OF THE PREFERRED EMBODIMENTS

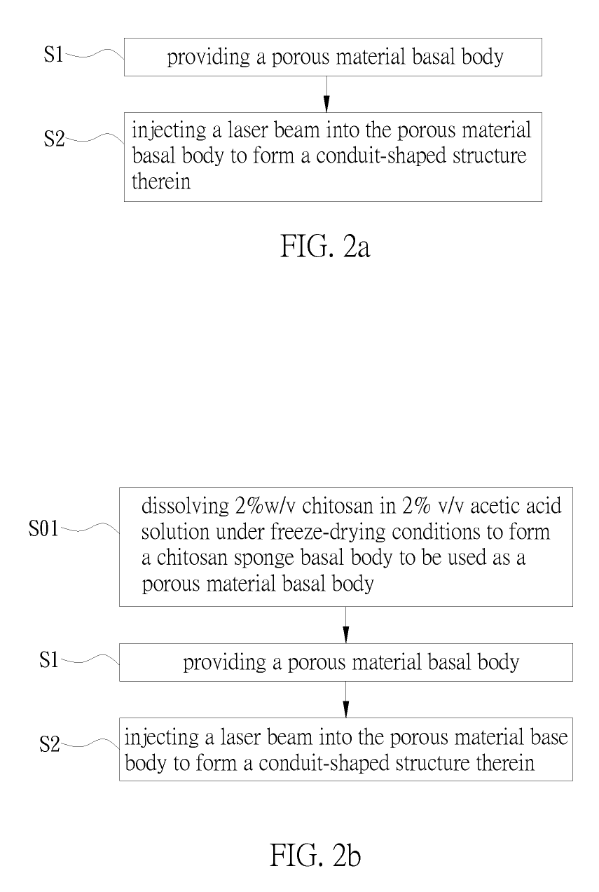

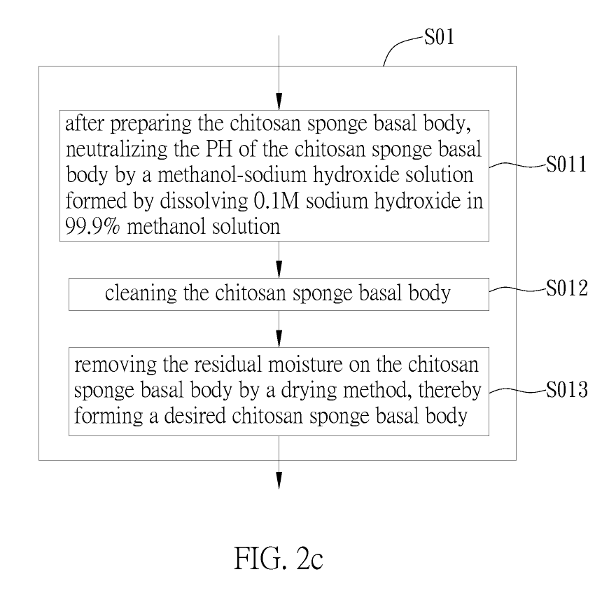

Illustrative embodiments of a porous material conduit and a method for preparing the same, of the present invention, are described as follows with reference to FIGS. 2a to 5. It should be understood that the drawings are simplified schematic diagrams only showing the components relevant to the present invention, and the layout of components could be more complicated in practical implementation.

Referring to FIG. 2a, FIG. 2a is a first flowchart showing a method of preparing a porous material conduit of the present invention. As shown in FIG. 2a, the method for preparing a periodic, non-linear optical element at least comprises the following steps.

In step S1, a porous material basal body is provided. In the embodiment, the porous material basal body is preferably a chitosan sponge basal body. It should be noted that in other embodiments, the porous material basal body may be one made of, for example, hyaluronic acid, collagen, gelatin, algin, polyanhydride, polycaprolacton, polylactic acid or a copolymer thereof. Then, step S2 is performed.

In step S2, a laser beam is injected into the porous material basal body, so as to form a conduit-shaped structure in the porous material basal body. In the embodiment, a carbon dioxide laser is applied, along with a lens module, to adjust the laser beam to focus the laser beam into the porous material basal body, thereby forming a conduit-shaped structure in the porous material basal body. In addition, the depth of focus and laser spot of the laser beam into the porous material basal body can be adjusted through the lens module, so as to adjust the cross-sectional shape and pattern of the conduit-shaped structure.

Further, referring to FIG. 2b, FIG. 2b is a second flowchart showing a method of preparing a porous material conduit of the present invention. As shown in FIG. 2b, in a preferred aspect of the embodiment, the method further comprises the following steps before step S1.

In step S01, in a freeze-drying system, 2% v/v chitosan is dissolved in 2% v/v acetic acid solution, and stands for 5 to 7 days to form a chitosan sponge basal body, which is further used as a porous material basal body.

Referring to FIG. 2c, FIG. 2c is a third flowchart showing a method of preparing a porous material conduit of the present invention. As shown in FIG. 2c, in another preferred aspect of the embodiment, step S01 further comprises the following steps S01.

In step S011, after the chitosan sponge basal body is formed, the desired pH of the chitosan sponge basal body is obtained by neutralizing with a methanol-sodium hydroxide solution formed by dissolving 0.1M sodium hydroxide in 99.9% methanol solution. Then, step S012 is performed.

In step S012, the chitosan sponge basal body is cleaned. Preferably, sodium hydroxide and/or acetate remained on the chitosan sponge basal body is cleaned by a methanol solution. Then, step S013 is performed.

In step S013, residual moisture on the chitosan sponge basal body is removed by a drying method to obtain a desired chitosan sponge basal body.

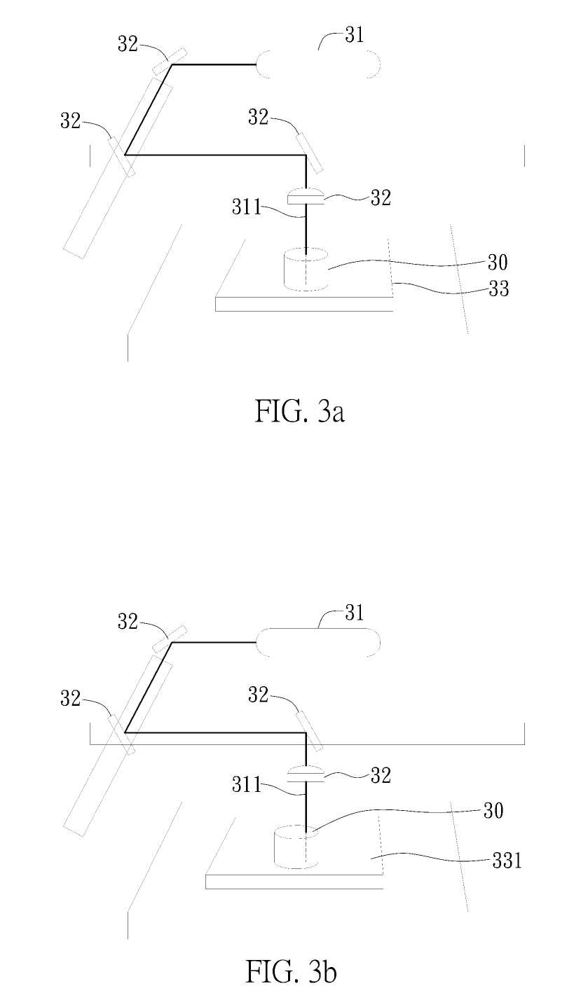

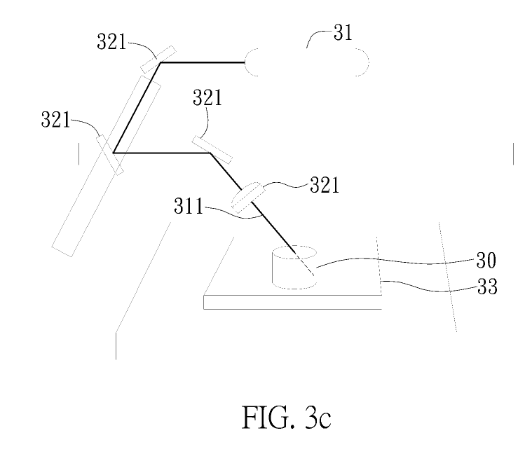

Moreover, referring to FIG. 3a, FIG. 3a is a first schematic diagram showing a device for preparing a porous material conduit of the present invention. As shown in FIG. 3a, the device comprises a laser source 31, a lens module 32, and a basal body fixing stand 33, wherein a laser beam 311 is obtained from the laser source 31 via the lens module 32.

In the embodiment, a porous material basal body 30 is first disposed on the basal body fixing stand 33, and then the laser beam 311 is controlled by the carbon oxide laser source 31, along with the lens module 31, so as to focus the laser beam 311 into the porous material basal body 30, thereby forming a conduit-shaped structure in the porous material basal body 30. In addition, the depth of focus or laser spot of the laser beam 311 can be adjusted through the lens module 30 to adjust the cross-sectional shape and pattern of the conduit-shaped structure.

Further, referring to FIG. 3b, FIG. 3b is a second schematic diagram showing a device for preparing a porous material conduit of the present invention. As shown in FIG. 3b, the device comprises the laser source 31, the lens module 32, and the basal body fixing stand 33, wherein the laser beam 311 is obtained from the laser source 31 through the lens module 32 that is capable of moving along different axes (such as, through a mechanical arm capable of moving along X-axis, Y-axis and/or Z-axis).

After the porous material basal body 30 is disposed on the basal body fixing stand 331, a two-dimensional or three-dimensional relative movement is generated between the movable fixing stand 331 carrying the porous material basal body 30 and the laser beam 311, and then the laser beam 311 is injected into the porous material basal body 30 to form another conduit-shaped structure, wherein the orginal conduit-shaped structure intersects with another conduit-shaped structure.

Similarly, if a plurality of relative movements is generated between the laser beam 311 and the porous material basal body 30 and the laser beam 311 is injected to the porous material basal body 30 during the intervals between the relative movements, a plurality of conduit-shaped structures can be formed in the porous material basal body 30. Preferably, if the relative movements are generated with same or equal intervals and the laser beam 311 is injected into the porous material basal body 30 during the intervals between the relative movements, a plurality of conduit-shaped structures parallel arranged into a two-dimensional array can be formed.

In addition, referring to FIG. 3c, FIG. 3c is a third schematic diagram showing a device for preparing a porous material conduit of the present invention. As shown in FIG. 3, the device comprises the laser source 31, the adjustable lens module 321, and the basal body fixing stand 33, wherein the laser beam 311 is obtained from the laser source 31 through the lens module 321.

After the porous material basal body 30 is disposed on the basal body fixing stand 33, the injection angle of the laser beam 311 with respect to the porous material basal body 30 is changed via the adjustable lens module 321, so as to form another conduit-shaped structure in the porous material basal body 30. As a result, the original conduit-shape structure intersects with another conduit-shaped structures.

Similarly, if the injection angle of the laser beam 311 with respect to the porous material basal body 30 is changed a plurality of times and the laser beam 311 is injected into the porous material basal body 30 a plurality of times during the changing of each of the angles, a plurality of intersecting, conduit-shaped structures can be formed in the porous material basal body 30.

More preferably, a plurality of relative movements is made between the laser beam 311 and the porous material basal body 30 via the adjustable lens group 321 and the basal body fixing station 331. Moreover, the injection angle of the laser beam 311 with respect to the porous material basal body 30 is changed during the relative movements, so as to form a plurality of conduit-shaped structures in the porous material basal body.

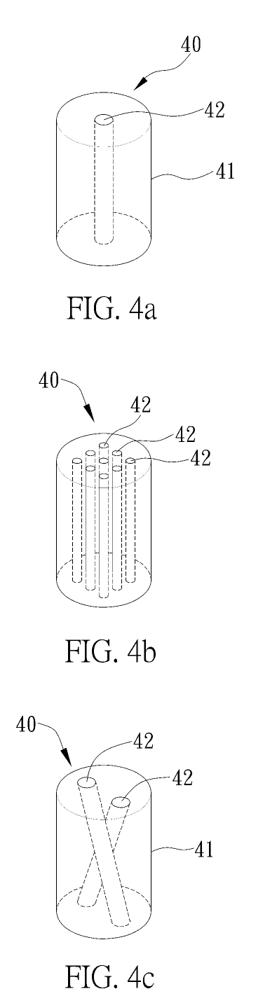

Referring to FIG. 4a, FIG. 4a is a first schematic diagram showing a porous material conduit of the present invention. As shown in FIG. 4a, the method for preparing the porous material conduit and the technology for manufacturing the device thereof, of the present invention, is used to prepare a porous material conduit 40, which comprises a porous material basal body 41 and a conduit-shaped structure 42 formed therein by laser.

Referring to FIGS. 4b and 4c, FIG. 4b and 4c are second and third schematic diagrams, respectively, showing a porous material conduit of the present invention. As shown in FIGS. 4b and 4c, a plurality of conduit-shaped structures 42 are formed, which can be arranged in an array or at least a pair thereof can be arranged in an intersecting alignment.

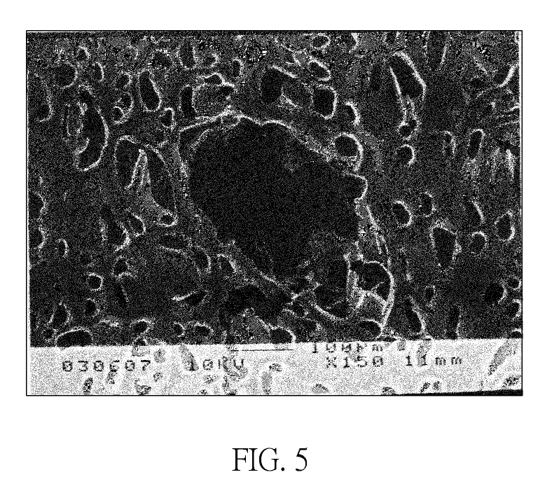

It should be noted that the technology disclosed herein for preparing the porous material conduit can prevent the structure of the porous material basal body from being damaged, as occurs when employing the conventional hole drilling technique. Hence, it is observable in FIG. 5 that under an electronic microscope, there is no collapse in the porous structure and bordering holes are open holes.

In conclusion, the porous material conduit and a method for preparing the same, as disclosed herein, can form a plurality of intersecting, hollow conduit-shaped structures in a porous material basal body by laser, while overcoming the drawback of the porous nerve conduit basal body being damaged as uneven clipping force or excessively large drilling force is applied in the conventional hole drilling technique.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangement.

What is claimed is:

1. A method for preparing a porous material conduit, comprising the steps of:
(1) providing a porous material basal body; and
(2) injecting a laser beam into the porous material basal body, so as to form a conduit-shaped structure in the porous material basal body.

2. The method for preparing a porous material conduit of claim 1, wherein the porous material basal body is made of one selected from the group consisting of a chitosan sponge basal body, hyaluronic acid, collagen, gelatin, algin, polyanhydride, polycaprolacton, polylactic acid and a copolymer thereof.

3. The method for preparing a porous material conduit of claim 1, wherein the porous material basal body is a chitosan sponge basal body.

4. The method for preparing a porous material conduit of claim 1, wherein the laser beam is a carbon dioxide laser.

5. The method for preparing a porous material conduit of claim 1, wherein the laser beam in the step (2), is focused into the porous material basal body via a lens module.

6. The method for preparing a porous material conduit of claim 1, further comprising a step (3) in which a relative movement is generated between the laser beam and the porous material basal body is generated, and then the laser beam is injected into the porous material basal body, so as to form another conduit-shaped structure in the porous material basal body, after the step (2) is performed.

7. The method for preparing a porous material conduit of claim 1, further comprising a step (3) in which an injection angle of the laser beam with respect to the porous material basal body is changed, so that the laser beam is injected into the porous material basal body, thereby forming another conduit-shaped structure in the porous material basal body, after the step (2) is performed.

8. The method for preparing a porous material conduit of claim 1, further comprising a step (3) in which a relative movement is generated between the laser beam and the porous material basal body, an injection angle of the laser beam with respect to the porous material basal body is changed, and then the laser beam injected into the porous material basal body, so as to form another conduit-shaped structure in the porous material basal body, after the step (2) is performed.

9. A porous material conduit, comprising:
a porous material basal body; and
a conduit-shaped structure formed in the porous material basal body by laser.

10. The porous material conduit of claim 9, wherein the porous material basal body is made of one selected from the group consisting of a chitosan sponge basal body, hyaluronic acid, collagen, gelatin, algin, polyanhydride, polycaprolacton, polylactic acid and a copolymer thereof.

11. The porous material conduit of claim 9, wherein the laser beam is a carbon dioxide laser.

12. The porous material conduit of claim 9, wherein structure collapse does not occur to a porous structure of the conduit-shaped structure.

13. The porous material conduit of claim 9, wherein bordering pores of the conduit-shaped structure are open pores.

14. A porous material conduit prepared according to the method of claim 1, comprising:
a porous material basal body; and
a plurality of conduit-shaped structures formed in the porous material basal body, wherein at least a pair of conduit-shaped structures have an intersecting alignment.

15. The porous material conduit of claim 14, wherein the porous material conduit is prepared according to the method for preparing a porous material conduit of claim 1.

16. A porous material conduit prepared using a method for preparing a porous material conduit, comprising the steps of:
a porous material basal body; and
a conduit-structure formed in the porous material basal body by laser.

17. The porous material conduit of claim 16, wherein the porous material basal body is made of one selected from the group consisting of a chitosan sponge basal body, hyaluronic acid, collagen, gelatin, algin, polyanhydride, polycaprolacton, polylactic acid and a copolymer thereof.

18. The porous material conduit of claim 16, wherein the laser beam is a carbon dioxide.

19. The porous material conduit of claim 16, wherein structure collapse does not occur to a porous structure of the conduit-shaped structure.

20. The porous material conduit of claim 16, wherein bordering pores of the conduit-shaped structure are open pores.
